(19) United States
(12) Patent Application Publication
(10) Pub. No.: US 2026/0365707 A1
(43) Pub. Date: Nov. 19, 2026

(54) IL-21 FUSION PROTEINS USEFUL AS ENHANCERS OF ANTI-CANCER IMMUNOTHERAPIES

(71) Applicant: ECOLE POLYTECHNIQUE FEDERALE DE LAUSANNE (EPFL), Lausanne (CH)

(72) Inventors: Yi WANG, Chavannes-pres-Renens (CH); Li TANG, Echichens (CH); Yugang GUO, Hangzhou (CN)

(21) Appl. No.: 19/113,472

(22) PCT Filed: Sep. 21, 2023
(86) PCT No.: PCT/EP2023/076099

§ 371 Date: Mar. 20, 2025

(30) Foreign Application Priority Data
Sep. 23, 2022 (EP) 22197346.4

(51) Int. Cl. C07K 14/54; A61K 35/17; A61K 38/00; A61P 35/00; C07K 14/765

(52) U.S. Cl. CPC C07K 14/54; A61K 35/17; A61P 35/00; C07K 14/765; A61K 38/00; C07K 2319/00; C07K 2319/30

(57) ABSTRACT

The present invention relates generally to the field of anti-cancer therapy, in particular to the use of agents or co-agents useful in anti-cancer immunotherapy such as adoptive T-cell transfer (ACT) immunotherapy and immune check-point blockades.

### FIELD OF THE INVENTION

The present invention relates generally to the field of anti-cancer therapy, in particular to the use of agents or co-agents useful in anti-cancer immunotherapy such as adoptive T-cell transfer (ACT) immunotherapy and immune check-point blockades.

### BACKGROUND OF THE INVENTION

Natural killer (NK) cell is a kind of cytotoxic lymphocytes belong to innate immune system with the capability of eliminating infections and cancerous cells, which are independent to the major histocompatibility complex (MHC) restriction (Wolf, N. K. et al., 2022) NK cell-based therapy is considered as an effective and safe treatment as accumulated impressive in vitro data reported on NK cell cytotoxicity towards cancers.

However, the clinical trial results are still unsatisfactory as it was found that during NK cells differentiation and maturation, they gain higher cytotoxicity although gradually loss stemness and homeostatic capability (Bald, T. et al. 2020) Particularly, in the tumor microenvironment (TME), the terminally differentiated NK cells are partially losing their killing capability and are unable to eliminate cancers (Li, Z.-Y. et al. 2021) The immune suppressive microenvironment hinders NK cell functions through various physiological factors, such as hypoxia, nutrition deprivation (Bi, J. & Tian, Z. 2017). Maintaining the NK cell sustainability in TME ensures the sustainable cytotoxic function towards cancer cells.

NK cell metabolism fulfills the biosynthetic and energy demands for survival, proliferation, and specialized functions (O'Brien, K. L. & Finlay, D. K. 2019). Therefore, it is a potential target that metabolically preserve the tumor infiltrated NK cells stem-like features to enhance their antitumor activity and therapeutic efficacy against cancers.

Interleukin-21 (IL-21) is a common cytokine receptor gamma-chain family involved in NK cells differentiation and regulation of their multiple functions (Parrish-Novak, J. et al. 2000). Here, we report that the engineered IL-21 (IL-21/Fc) retained NK cell stemness and restored their antitumor activity in TME, leading to enhanced efficacy against established solid tumors in multiple syngeneic tumors bearing mouse models when combined with IL-15SA treatment.

Thus, the development of engineering processes and molecules supporting immune cell metabolic fitness, expansion, and survival within the TME, as well as effective immune cells with enhanced antitumor activity, are still urgently needed.

### SUMMARY OF THE INVENTION

The present invention provides a fusion protein for use in the prevention and/or treatment of a cancer, wherein said fusion protein comprises (i) an immunoglobulin IgG Fc domain or a Human serum albumin (HSA) polypeptide, and (ii) a polypeptide comprising a sequence of an Interleukin-21 (IL-21) polypeptide, a fragment or a variant thereof, wherein the IL-21 polypeptide, fragment, or variant thereof, is covalently fused to the N-terminus or the C-terminus of the IgG Fc domain or the HAS polypeptide via a linker.

Further provided is a fusion protein comprising (i) an immunoglobulin IgG Fc domain or Human serum albumin (HSA) polypeptide, and (ii) a polypeptide comprising a sequence of an Interleukin-21 (IL-21) polypeptide, a fragment or a variant thereof, wherein the IL-21 polypeptide, fragment, or variant thereof, is covalently fused to the N-terminus or the C-terminus of the IgG Fc domain or the HAS polypeptide via a linker.

Further provided is a nucleic acid encoding one or more fusion protein(s) according to the invention.

Further provided is a plasmid or a vector comprising a nucleic acid according to the invention.

Also provided is an isolated host cell, or population of cells, comprising the plasmid or vector according to the invention.

Also provided is a pharmaceutical composition comprising i) a fusion protein for use according to the invention, ii) a fusion protein according to the invention, iii) a plasmid or a vector according to the invention, or iv) an isolated host cell, or population of cells according to the invention, and a pharmaceutically acceptable carrier, a diluent and/or an excipient.

Also provided are methods of treatment and/or prevention of a cancer in a subject comprising administering the pharmaceutical composition of the invention.

Also provided are methods of treatment and/or prevention comprising (i) removing and isolating immune cells, preferably native T cells, from said patient or subject, (ii) genetically engineering said T cells to encode a chimeric antigen receptor (CAR), a T cell receptor (TCR) or any other synthetic tumor targeting motif or antigen, (iii) expanding ex vivo into a larger population of engineered T cells, and (iv) reintroducing said engineered T cells, into the patient or subject.

Also provided is a method of enhancing ACT antitumor activity in a subject comprising administering the pharmaceutical composition of the invention.

### BRIEF DESCRIPTION OF THE FIGURES

FIG. 1. Schematic representation of a homodimerized IL-21/IgG Fc fusion protein.

FIG. 2. IL-21/Fc production and function characterization. a. Sodium dodecyl sulfate-polyacrylamide gel electrophoresis (SDS-PAGE) analysis of purified IL-21/Fc. βME, β-mercaptoethanol. b. Activated NK cells were cultured in presence of IL-21 or IL-21/Fc for 24 h at indicated concentrations. The fold changes of IFN-γ expression of NK cells were normalized to NK cells treated with PBS. c-d. Pharmacokinetic profile and half-life time of IL-21 or IL-21/Fc. Blood from C57/BL6J mice were collected at indicated time points after a single intravenous injection (n=5, data are mean±SEM), IL-21 concentrations were measured by ELISA. e. Killing efficacy of activated NK cells to B16F10_β2m (left) and CT26_β₂m (right) cells at the E:T ratio to 0.5 in the presence of IL-21/Fc at 100 ng/ml (n=5, data are mean±SEM). f. Mean fluorescence intensity of CD107a, granzyme B, and IFN-γ expression of activated NK cell in presence of IL-21/Fc at 100 ng/ml.

FIG. 3. IL-21/Fc inhibits tumor growth and facilitate IL-15SA antitumor activity. a. Experiment timeline. Mice were subcutaneously (s.c.) inoculated with B16F10_β₂m melanoma cells (5×10⁵/mouse), CT26_β₂m colon carcinoma cells (5×10⁵/mouse), or RMA-S lymphoma cells (8×10⁵/mouse). Mice received treatments from day 7, Eight times of IL-21/Fc (20 μg/mouse) were intratumorally (i.t.) injected to mice every other day, and/or twice of IL-15SA (5 μg/mouse) were i.t. injected to mice every week, PBS were i.t. injected as control. b-d. Average tumor area and survival curves of mice bearing with B16F10_β₂m melanoma (left), CT26_β₂m colon carcinoma (middle), and RMA-S lymphoma (right) (n=8, data are mean±SEM).

FIG. 4. IL-21 combination therapy suppresses tumor growth. IL-21/Fc combined with other therapies (other than ACT) for cancer immunotherapy shows that the fusion protein enhances various immunotherapies. Mice were subcutaneously inoculated with B16F10 melanoma cells (5×10⁵), followed by 8 doses of IL-21/Fc (20 μg) were injected every two days from day 7 in addition to combination treatment, i.t. administration of IL-15SA (5 μg) on day 7 and day 14, 3 doses of anti-PD-1 (100 μg) were i.p. injected every three days from day 7, s.c. administration of 8 doses of R837 (1 mg/kg) from day 7 for every two days. Average tumor area and survival curves of mice bearing B16F10 melanoma (n=5, data are mean±SEM).

FIG. 5. Antitumor activity of IL-21/Fc is dependent on NK cells in tumor. a. Tumor infiltrated immune cell subsets analysis. CT26_β₂m tumor bearing BALB/c mice received IL-21/Fc (i.t. 20 μg/mouse) every two days, and/or IL-15SA (5 μg/mouse) every week from day 7. Tumors were excised one week after treatment and the tumor infiltrated CD45+ lymphocytes (TILs), NK cells, CD8+ T cells, CD4+ T cells, B cells, dendritic cells (DC), macrophages, neutrophils, and eosinophils were analyzed by FACS. b. Average tumor area of B16F10_β₂m tumor bearing C57/BL6J mice. Mice received injections from day 7, eight times of IL-21/Fc (i.t. 20 μg/mouse) every, twice IL-15SA (5 μg/mouse) every week. Three times of anti-NK1.1 (PK136, BioXcell, 400 μg/mouse) were intraperitoneally (i.p.) injected one day before treatment, every four days (n=5, data are mean±SEM).

FIG. 6. IL-21/Fc enhances NK cell function by promoting glycolytic metabolism. a. Real-time analysis of extracellular acidification rate (ECAR). b. Average basal glycolysis, glycolytic reserved capacity, and the ratio of OCR (oxygen consumption rate) to ECAR of activated NK cells in presence of IL-21/Fc after 24 hours incubation. c. Glycolysis associated gene expression changes of activated NK cells in presence of IL-21/Fc after 24 hours incubation. d. Ratio of frequencies changes of granzyme B+ and IFN-γ+ of activated NK cells in presence of IL-21/Fc and indicated inhibitors after 24 hours incubation, 2-DG, 2 mM; Oligomycin 1 μM. e-f. CT26_β₂m tumor bearing BALB/c mice received IL-21/Fc (i.t. 20 μg/mouse) every two days, and/or IL-15SA (5 μg/mouse) every week from day 7. Tumors were excised one week after treatment and analyzed by FACS. Frequencies of glucose transporter 1 (Glut1) expression and MFI of 2-NBDG uptake cells (e), frequencies of granzyme B, IFN-γ and TNF-α expression (f) of tumor infiltrated NKp46+ TILs.

FIG. 7. IL-21/Fc enriches NK cells with promoted stemness and induce durable protection again tumor rechallenge. a. CT26_β2m tumor bearing BALB/c mice received IL-21/Fc (i.t. 20 μg/mouse) every two days, and/or IL-15SA (5 μg/mouse) every week from day 7. Spleens and tumors were excised one week after treatment and analyzed by FACS. Representative flow cytometry plots showing the frequencies of double negative (DN), CD27+, and CD11b+ NK cell population among all CD45.2+NKp46+ cells in spleen (up) or tumor (bottom). b. Percentage of CD11b+ cells and CD27+ cells among CD45.2+NKp46+ cells in spleen or tumor. c. Frequencies of Granzyme B+, IFN-γ+, and TNF-α+ among NKp46+ TILs. d. Frequencies of CD27+, Sca1+ population in CD45.2+NKp46+ tumor infiltrated cells; MFI of TCF1 and Ki67 in NKp46+ TILs. e. Survival curves and numbers of long-term surviving mice against the re-challenges. CT26_β₂m tumor bearing BALB/c mice or B16F10_β₂m tumor bearing C57BL6 mice received IL-21/Fc (i.t. 20 μg/mouse) every two days, IL-15SA (5 μg/mouse) every week. Survivors from combination groups were rechallenged s.c. by CT26_β₂m or B16F10_β₂m respectively on day 90 post primary inoculation.

FIG. 8. Human IL-21/Fc promotes human NK cells function and glycolysis. a. Sodium dodecyl sulfate-polyacrylamide gel electrophoresis (SDS-PAGE) analysis of purified human IL-21/Fc (hIL-21/Fc). βME, β-mercaptoethanol. b. Killing efficacy of activated NK92MI cells to K562 cells at indicated E:T ratios in the presence of hIL-21/Fc at 100 ng/ml (n=5, data are mean±SEM). c. Basal glycolysis of NK92MI cells in presence of hIL-21/Fc at 100 ng/ml after 24 h incubation.

FIG. 9. IL-21/Fc enhances the killing efficiency of PBMC derived human NK cells. Human NK cells were isolated from Peripheral blood mononuclear cells (PBMC) through magnetic-activated cell sorting (MACS). Isolated human NK cells were activated and cultured in presence of human IL-21 (50 U/ml) for 5 days. Killing efficacy of activated NK cells towards indicated target cells in presence of hIL-21/Fc (100 ng/mL) with E/T ratio at 0.5 after 5 hours incubation.

FIG. 10. IL-21/Fc promotes the antitumor efficacy of transferred human NK92MI cells against the K562 human lymphoma in a xenograft model. Immunodeficient NSG mice were subcutaneously inoculated with K562 lymphoma cells (8×105). NK92MI (3×106) were transferred to tumor bearing mice on day 7 followed by 8 doses of IL-21/Fc (20 μg) that were injected every two days. Average tumor area and survival curves of mice bearing K562 lymphoblast tumors (n=7, data are mean±SEM).

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein, “at least one” means “one or more”, “two or more”, “three or more”, etc. As used herein the terms “subject”/“subject in need thereof”, or “patient”/“patient in need thereof” are well-recognized in the art, and, are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. However, in other aspects, the subject can be a normal subject. The term does not denote a particular age or sex. Thus, adult and newborn subjects, whether male or female, are intended to be covered. Preferably, the subject is a human, most preferably a human suffering from cancer and/or cancer metastasis or a human that might be at risk of suffering from cancer and/or cancer metastasis. The cancer is a solid cancer or a liquid cancer.

In one aspect, the solid cancer is selected from the non-limiting group comprising lung cancer, breast cancer, ovarian cancer, cervical cancer, uterus cancer, head and neck cancer, glioblastoma, hepatocellular carcinoma, colon cancer, rectal cancer, colorectal carcinoma, kidney cancer, prostate cancer, gastric cancer, bronchus cancer, pancreatic cancer, urinary bladder cancer, hepatic cancer and brain cancer and skin cancer, in particular melanoma, or a combination of one or more thereof.

In one specific aspect, the cancer is an MHC-I expression altered cancer, preferably an MHC-I deficient solid cancer.

The terms “nucleic acid”, “polynucleotide,” and “oligonucleotide” are used interchangeably and refer to any kind of deoxyribonucleotide (e.g. DNA, cDNA, . . . ) or ribonucleotide (e.g. RNA, mRNA, . . . ) polymer or a combination of deoxyribonucleotide and ribonucleotide (e.g. DNA/RNA) polymer, in linear or circular conformation, and in either single or double stranded form. These terms are not to be construed as limiting with respect to the length of a polymer and can encompass known analogues of natural nucleotides, as well as nucleotides that are modified in the base, sugar and/or phosphate moieties (e.g. phosphorothioate backbones). In general, an analogue of a particular nucleotide has the same base-pairing specificity, i.e., an analogue of A will base-pair with T.

The term “vector”, as used herein, refers to a viral vector or to a nucleic acid (DNA or RNA) molecule such as e.g., a plasmid or other vehicle, which contains one or more heterologous nucleic acid sequence(s) of the invention and, preferably, is designed for transfer between different host cells. The terms “expression vector”, “gene delivery vector” and “gene therapy vector” refer to any vector that is effective to incorporate and express one or more nucleic acid(s) of the invention, in a cell, preferably under the regulation of a promoter. A cloning or expression vector may comprise additional elements, for example, regulatory and/or post-transcriptional regulatory elements in addition to a promoter.

The term “about,” particularly in reference to a given quantity, number or percentage, is meant to encompass deviations of plus or minus ten percent (±10). For example, about 5% encompasses any value between 4.5% to 5.5%, such as 4.5, 4.6, 4.7, 4.8, 4.9, 5, 4.1, 5.2, 5.3, 5.4, or 5.5.

As used herein, Interleukine-21 (IL-21), refers to a member of the IL-21 family cytokines. IL-21) is a common cytokine receptor gamma-chain family involved in NK cells differentiation and regulation of their multiple functions (O'Brien, K. L. & Finlay, D. K. 2019). “IL-21, a fragment or a variant thereof” include sequences comprising the sequence of, preferably, native human IL-21 as well as fragment and variants thereof. In one aspect, the IL-21 sequence is a human IL-21 amino acid sequence as set forth in SEQ ID No. 1.

The term “variant”, when it refers to IL-21, means one or more biologically active derivatives of an IL-21, preferably of a human IL-21 sequence of the invention. In general, the term “variant” refers to molecules having a native sequence with one or more additions, substitutions (generally conservative in nature) and/or deletions, relative to the native molecule, so long as the modifications do not destroy its biological activity and which are “substantially homologous” to the reference molecule (Gorby et al., Sci. Signal. 13, eabc0653, 2020; Saxton et al., Science 371, eabc8433, 2021). In general, the sequences of such variants will have a high degree of sequence homology or identity to the reference sequence, e.g., sequence homology or identity of more than 25%, generally more than 50% to 70%, even more particularly 80%, or 85% or more, such as at least 90%, or 95% or more, when the two sequences are aligned. Preferably, the reference sequence is the human IL-21 amino acid sequence as set forth in SEQ ID No. 1.

As used herein, a “fragment” of an IL-21, preferably of a human IL-21, of the invention refers to a sequence containing less amino acids or nucleotides in length than the respective polypeptide sequence or nucleic acid sequence. Preferably, this polypeptide sequence or fragment contains less than 90%, preferably less than 60%, in particular less than 30% nucleotides in length than the respective polypeptide sequence or nucleic acid sequence, e.g. human IL-21 amino acid sequence as set forth in SEQ ID No. 1.

While focusing on developing novel and efficient approaches for treating tumors, the Inventors have surprisingly shown that engineered IL-21/IgG Fc fusion protein (IL-21/Fc) showed increased half-life time, promote NK cell cytotoxicity and stemness through metabolic modulation and thus enhance the efficacy of NK cells against established tumors, especially MHC-I deficient tumors. These promising results provide a novel and efficient approach that will overcome a major barrier in the current NK cell-based immunotherapy in the clinic and increase the response rate.

The present invention provides, in one aspect, a fusion protein comprising (i) a polypeptide comprising a sequence of an Interleukin-21 (IL-21) polypeptide, a fragment or a variant thereof and (ii) a molecule that increases the half-life time of said IL-21 polypeptide, fragment or variant thereof.

As used herein, the molecule that increases the half-life time of an IL-21 polypeptide, fragment or variant thereof is preferably selected from the group comprising a Fc domain of an IgG and a Human serum albumin (HSA) such as e.g., the sequence comprising or consisting of, SEQ ID No. 17, a fragment or variant of any one thereof.

As used herein, the Fc domain of an IgG is preferably a silent Fc domain of an immunoglobulin (Ig) G, preferably of a mouse or a human IgG, most preferably of a human IgG1, IgG2, IgG3 or IgG4, a fragment or a variant thereof. In one aspect, the Fc domain of a human IgG is selected from the group comprising a sequence comprising, or consisting of, IgG1 Fc (SEQ ID No. 2), IgG2 Fc (SEQ ID No. 8), IgG3 Fc (SEQ ID No. 11), and IgG4 Fc (SEQ ID No. 14), a fragment, a variant, or a combination of one or more of these sequences.

In one aspect, the IL-21, fragment, or variant thereof, is covalently fused to the N-terminus or the C-terminus of the Fc domain or the HAS polypeptide by, or via a linker, e.g. a polypeptide linker. In one aspect, the polypeptide linker consists primarily of stretches of Gly and Ser residues (“GS” linker) or Gly-Gly and Ser residues (“GGS” linker) followed or not by one or more Arg residue (“R” residue”). Usually, the linkers comprise 10-30 amino-acids, preferably, 10-25 amino-acids, and more preferably 15-25 amino-acids. Non-limiting examples of GGS and GGGGS linkers are disclosed herein.

According to one aspect, the IgG Fc domain can be an Fc domain obtained from mouse IgG1, IgG2a, IgG2b and/or IgG3 isoform, or a variant of said fragment.

According to one aspect, the IgG Fc domain can be an Fc domain obtained from human IgG1, IgG2, IgG3 and/or IgG4 isoform, or a variant of said fragment.

In one aspect, the IgG Fc domain of the fusion protein dimerizes with a second IgG Fc domain thereby forming a homodimer wherein the second IgG Fc domain is covalently fused, via its N-terminus or C-terminus, to an IL-21 polypeptide, fragment, or variant thereof. This homodimerization is done through non-covalent binding between the first IgG Fc domain and the second IgG Fc domain, thereby enhancing the half-life of the fusion protein. Usually, the first and the second IgG Fc domain are similar (e.g. two human IgG1, two human IgG 2, . . . ).

The term “variant”, when it refers to an IgG Fc fragment, means one or more biologically active derivatives of an IgG Fc fragment, preferably of a human IgG Fc fragment sequence of the invention. In general, the term “variant” refers to molecules having a native sequence with one or more additions, substitutions (generally conservative in nature) and/or deletions, relative to the native molecule, so long as the modifications do not destroy its biological activity, and which are “substantially homologous” to the reference molecule. In general, the sequences of such variants will have a high degree of sequence homology or identity to the reference sequence, e.g., sequence homology or identity of more than 25%, generally more than 50% to 70%, even more particularly 80%, or 85% or more, such as at least 90%, or 95% or more, when the two sequences are aligned. Preferably, the reference sequence is the human IgG Fc fragment amino acid sequence as set forth in any one of sequences IgG1 Fc (SEQ ID No. 2), IgG2 Fc (SEQ ID No. 8), IgG3 Fc (SEQ ID No. 11), and IgG4 Fc (SEQ ID No. 14), a fragment or a combination of one or more of these sequences.

A variant of an IgG Fc fragment can be mutated, e.g. for decreasing the antibody-dependent cell-mediated cytotoxicity (ADCC) such as described in Czajkowsky et al., 2012, EMBO Mol. Med, 1015-1028 or for increasing half-life or in vivo level of IgG as described in Zalevsky et al., 2010, Nat. Biotechnol. 28, 157-159; Vaccaro et al., 2005, Nat. Biotechnol. 23, 1283-1288 (e.g. IL-10/Fc).

Point mutations can be introduced in an IgG Fc fragment, such as e.g., an IgG1 Fc domain as described in Armour et al., 1999, Eur. J. Immunol. 29, 2613-2624 or Zheng X X et al., 1995, J. Immunol. 154, 5590-5600 to generate a non-cytolytic IgG1 Fc domain.

According to an aspect of the invention, the variant of SEQ ID No: 2 comprises at least one mutation selected from C220A, L234A, L235A and P329G. In a further aspect of the invention, the variant of SEQ ID No: 2 comprises at least two, at least three, or at least four mutations selected from C220A, L234A, L235A and P329G. The positions of these mutations are indicated with reference to the full human IgG1 sequence.

According to an aspect of the invention, the variant of SEQ ID No: 8 comprises at least one mutation selected from A330S and P331S. In a further aspect of the invention, the variant of SEQ ID No: 8 comprises at least two mutations selected from A330S and P331S. The positions of these mutations are indicated with reference to the full human IgG2 sequence.

According to an aspect of the invention, the variant of SEQ ID No: 14 comprises at least one mutation selected from S228P and L235E. In a further aspect of the invention, the variant of SEQ ID No: 14 comprises at least two mutations selected from S228P and L235E. The positions of these mutations are indicated with reference to the full human IgG4 sequence.

According to a particular aspect, Fc fusion proteins as well as HSA fusion proteins of the invention can alternatively be modified for further extending its half-life in vivo by standard strategies, including pegylation (e.g. pegylation of the human IL-21 sequence, fragment or variant thereof: such as described in Mumm et al., 2011) The Fc domain of Fc fusion protein IL-21/Fc of the invention can also been replaced by antibodies or human serum albumin or variant thereof, such as described or reviewed in Qiao, et al., 2019, Cancer Cell 35, 901-915.e4; Kontermann, 2011, Curr. Opin. Biotechnol., 22, 868-876).

In one aspect, the IL-21 is a mouse IL-21 sequence that comprises or consists of SEQ ID No. 20, a fragment or a variant thereof.

In one aspect, the IL-21 is a human IL-21 sequence that comprises or consists of SEQ ID No. 1, a fragment or a variant thereof.

Non-limiting examples of the fusion proteins of the invention comprise, or consist of, SEQ ID No. 3, SEQ ID No. 6, SEQ ID No. 7, SEQ ID No. 9, SEQ ID No. 10, SEQ ID No. 12, SEQ ID No. 13, SEQ ID No. 15, SEQ ID No. 16, SEQ ID No. 18, SEQ ID No. 19, and SEQ ID No. 22, a fragment or a variant of any one of the sequences thereof.

The term “variant”, when it refers to a fusion protein, means one or more biologically active derivatives of a fusion protein, preferably of a sequence described in the invention. In general, the term “variant” refers to molecules having a native sequence with one or more additions, substitutions (generally conservative in nature) and/or deletions, relative to the native molecule, so long as the modifications do not destroy its biological activity and which are “substantially homologous” to the reference molecule (Gorby et al., Sci. Signal. 13, eabc0653, 2020; Saxton et al., Science 371, eabc8433, 2021). In general, the sequences of such variants will have a high degree of sequence homology or identity to the reference sequence, e.g., sequence homology or identity of more than 25%, generally more than 50% to 70%, even more particularly 80%, or 85% or more, such as at least 90%, or 95% or more, when the two sequences are aligned.

As used herein, a “fragment” of a fusion protein of the invention refers to a sequence containing less amino acids in length than the respective polypeptide sequence. Preferably, this sequence or fragment contains less than 90%, preferably less than 60%, in particular less than 30% nucleotides in length than the respective polypeptide sequence, as described herein.

In one aspect, the fusion protein described herein is for use in the prevention and/or treatment of a cancer. Preferably, the cancer is a solid cancer or a liquid cancer. More preferably, the cancer is a solid cancer selected from the group comprising lung cancer, breast cancer, ovarian cancer, cervical cancer, uterus cancer, head and neck cancer, glioblastoma, hepatocellular carcinoma, colon cancer, rectal cancer, colorectal carcinoma, kidney cancer, prostate cancer, gastric cancer, bronchus cancer, pancreatic cancer, urinary bladder cancer, hepatic cancer and brain cancer and skin cancer, in particular melanoma, or a combination of one or more thereof.

In one aspect, the cancer is an MHC-I expression altered cancer (e.g. downregulation or complete loss of the MHC-I expression), preferably an MHC-I deficient solid cancer. The alteration of MHC class I (MHC-I) expression is a frequent event during cancer progression, allowing tumor cells to evade the immune system. MHC-I expression altered cancers are, e.g. discussed in Cornel, A. M., Mimpen, I. L., & Nierkens, S. (2020). MHC Class I Downregulation in Cancer: Underlying Mechanisms and Potential Targets for Cancer Immunotherapy. Cancers, 12(7), 1760.

In one aspect, the fusion protein described herein is used in combination with another cancer therapy. Preferably, the other cancer therapy is an anti-cancer immunotherapy selected from the group comprising ACT therapy, immune checkpoint blockade therapy, cytokine therapy, cancer vaccine therapy, bispecific antibody therapy and other cancer immunotherapies (such as e.g., chemotherapy, radiotherapy and hormonotherapy), or a combination of one or more thereof.

A chemotherapy of the present invention can concern agents that damage DNA and/or prevent cells from multiplying, such as genotoxins.

Genotoxins can be selected from the group comprising alkylating agents, antimetabolites, DNA cutters, DNA binders, topoisomerase poisons and spindle poisons. Examples of alkylating agents are lomustine, carmustine, streptozocin, mechlorethamine, melphalan, uracil nitrogen mustard, chlorambucil, cyclosphamide, iphosphamide, cisplatin, carboplatin, mitomycin, thiotepa, dacarbazin, procarbazine, hexamefhyl melamine, triethylene melamine, busulfan, pipobroman, mitotane and other platine derivatives.

An example of DNA cutters is bleomycin.

Topoisomerases poisons can be selected from the group comprising topotecan, irinotecan, camptothecin sodium salt, daorubicin, doxorubicin, idarubicin, mitoxantrone teniposide, adriamycin and etoposide.

Examples of DNA binders are dactinomycin and mithramycin whereas spindle poisons can be selected among the group comprising vinblastin, vincristin, navelbin, paclitaxel and docetaxel.

A chemotherapy of the present invention can concern antimetabolites selected among the following compounds: methotrexate, trimetrexate, pentostatin, cytarabin, ara-CMP, fludarabine phosphate, hydroxyurea, fluorouracyl, fioxuridine, chlorodeoxyadenosine, gemcitabine, thioguanine and 6-mercaptopurine.

Radiotherapy refers to the use of high-energy radiation to shrink tumors and kill cancer cells. Examples of radiation therapy include, without limitation, external radiation therapy and internal radiation therapy (also called brachytherapy).

External radiation therapy is most common and typically involves directing a beam of direct or indirect ionizing radiation to a tumor or cancer site. While the beams of radiation, the photons, the Cobalt or the particle therapy are focused to the tumor or cancer site, it is nearly impossible to avoid exposure of normal, healthy tissue. Energy source for external radiation therapy is selected from the group comprising direct or indirect ionizing radiation (for example: x-rays, gamma rays and particle beams or combination thereof).

Internal radiation therapy involves implanting a radiation-emitting source, such as beads, wires, pellets, capsules, etc., inside the body, at, or near to the tumor site. Energy source for internal radiation therapy is selected from the group of radioactive isotopes comprising: iodine (iodine125 or iodine131), strontium89, radioisotopes of phosphorous, palladium, cesium, indium, phosphate, or cobalt, and combination thereof. Such implants can be removed following treatment, or left in the body inactive. Types of internal radiation therapy include, but are not limited to, interstitial, and intracavity brachytherapy (high dose rate, low dose rate, pulsed dose rate).

A currently less common form of internal radiation therapy involves biological carriers of radioisotopes, such as with radio-immunotherapy wherein tumor-specific antibodies bound to radioactive material are administered to a patient or subject. The antibodies bind tumor antigens, thereby effectively administering a dose of radiation to the relevant tissue.

Methods of administering radiation therapy are well known to those of skill in the art.

A variety of other additional therapeutic agents may be used in conjunction with the compositions described herein.

Additional therapeutic agents suitable for use in combination with the invention include, but are not limited to, ibrutinib (Imbruvica®), ofatumumab (Arzerra®), rituximab (Rituxan®), bevacizumab (Avastin®), trastuzumab (Herceptin®), trastuzumab emtansine (KADCYLA®), imatinib (Gleevec®), cetuximab (Erbitux®), panitumumab (Vectibix®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In additional aspects, the additional therapeutic agent can be an anti-inflammatory agent. Anti-inflammatory agents or drugs include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

ACT therapy, as used herein, is selected from the group non-limiting comprising non-limiting group comprising T cell, chimeric antigen receptor (CAR)-T cell, T cell receptor (TCR)-transgenic T cell, tumor infiltrating lymphocyte (TIL), NK cell, NK-T cell, CAR-NK cell, CAR-NKT cell, TCR-transgenic NK cell, TCR-transgenic NK-T cell, dendritic cell, macrophage, CAR-macrophage or any synthetic tumor specific immune cells. In a preferred aspect, the ACT therapy is selected from the group comprising TCR-T, CAR-T, TILs and NK cell therapy/ies, or a combination of one or more thereof.

Non-limiting examples of ACT immunotherapies are listed in Fan et al., 2018, Theranostics, 8(20): 5784-5800; Rosenberg et al., 2008, Nat. Rev. Cancer 8, 299-308.

Cytokine therapy, as used herein, is selected from the non-limiting group comprising GM-CSF, IFN gamma, IL-7, IL-10, IL-12, IL-15, and a fusion protein thereof comprising a cytokine and an immunoglobulin IgG Fc domain, human serum albumin (HAS) or a combination of one or more thereof.

In one aspect, Cytokine therapy, is selected from the non-limiting group comprising IL-15, a fusion protein comprising IL-15, IL-15Ra and an immunoglobulin IgG Fc domain, or a combination of one or more thereof.

In a preferred aspect, the fusion protein comprising IL-15, IL-15Ra and an immunoglobulin IgG Fc domain is IL-15 superagonist (see e.g. Karin M. Knudson et al., Expert Opin Biol Ther. 2020).

NK cell therapy, as used herein, is selected from the non-limiting group comprising induced or activated NK cells, iPSC-NK cells, hESCs-NK cells, CAR-NK cells, CB-NK cells and PBNK cells, or a combination of one or more thereof.

NK cells can be autologous or allogeneic NK cells.

Induced or activated NK cells refer to cells that have been cultivated and expanded, usually in-vitro, in the presence of IL-2 and further maintained until being (re)-injected in the patients in need thereof.

iPSC-NK cells refer to NK cells derived from induced pluripotent stem cells.

hESCs-NK cells refer to NK cells derived from human embryonic stem cells.

CAR-NK cells refer to NK cells engineered to express chimeric antigen receptors (CARs) (see e.g. Daher M, et al. Clin Transl Immunology. 2021 Apr. 28; 10(4)).

PBNK cells or PB-NK cells refer both to peripheral blood NK cells that are collected from a donor by apheresis and expanded prior to use (Fujisaki H, et al., Cancer Res. 2009).

CB-NK cells are usually obtained from an umbilical cord blood unit and expanded (Shah N, et al., PloS One 2013).

Any sources of NK cells are considered in the present invention. Currently, clinical-grade NK cells can be manufactured in a large scale from multiple sources including NK92 cell line, peripheral blood mononuclear cells (PBMCs), umbilical cord blood cells (UBC), CD34+ hematopoietic progenitor cells (HPCs), and induced pluripotent stem cells (iPSCs).

Immune checkpoint blockade therapy, as used herein, comprises inhibitors selected from the group comprising a CTLA-4 inhibitor, a TIM3 inhibitor, a PD-1 inhibitor, a TIGIT inhibitor, a LAG-inhibitor, and a PD-L1 inhibitor, or a combination of one or more thereof.

Non-limiting examples of PD-1 inhibitors comprise inhibitors such as nivolumab (Opdivo®), pembrolizumab (Keytruda®), pembrolizumab, pidilizumab, and atezolizumab.

Non-limiting examples of PD-L1 inhibitors comprise inhibitors such as atezolizumab, avelumab, AMP-224, MEDI-0680, RG-7446, GX-P2, durvalumab, KY-1003, KD-033, MSB-0010718C, TSR-042, ALN-PDL, STI-A1014, CX-072, and BMS-936559.

Non-limiting examples of CTLA-4 inhibitors include ipilimumab (Yervoy) (also known as BMS-734016, MDX-010, MDX-101) and tremelimumab (formerly ticilimumab, CP-675,206).

Non-limiting examples of TIGIT inhibitors include Tiragolumab (MTIG7192A; RG6058), AB154 (Arcus Biosciences), MK-7684 (Merck), BMS-986207 (Bristol-Myers Squibb), ASP8374 (Astellas Pharma) and ASP8374 (Astellas Pharma).

In one aspect, the fusion protein for use described herein, such as e.g. an IL-21-Fc fusion protein, increases the efficacy of the anti-cancer immunotherapy of an increase equal or superior to about 2%, equal or superior to about 5%, equal or superior to about 20%, equal or superior to about 40%, equal or superior to about 60%, equal or superior to about 500%, when compared to the efficacy of the anti-cancer therapy in the absence of the fusion protein (e.g. in the absence of the Fc-IL-21 fusion protein).

Further provided is a nucleic acid sequence encoding one or more recombinant constructs of the invention, such as e.g., the fusion proteins of the invention. Non-limiting examples of the fusion proteins of the invention comprise, or consist of, SEQ ID No. 3, SEQ ID No. 6, SEQ ID No. 7, SEQ ID No. 9, SEQ ID No. 10, SEQ ID No. 12, SEQ ID No. 13, SEQ ID No. 15, SEQ ID No. 16, SEQ ID No. 18, SEQ ID No. 19, and SEQ ID No. 22, a fragment or a variant of any one of the sequences thereof.

In general, the term “variant” refers to molecules having a native sequence with one or more additions, substitutions (generally conservative in nature) and/or deletions, relative to the native molecule, so long as the modifications do not destroy its biological activity and which are “substantially homologous” to the reference molecule (Gorby et al., Sci. Signal. 13, eabc0653, 2020; Saxton et al., Science 371, eabc8433, 2021). In general, the sequences of such variants will have a high degree of sequence homology or identity to the reference sequence, e.g., sequence homology or identity of more than 25%, generally more than 50% to 70%, even more particularly 80%, or 85% or more, such as at least 90%, or 95% or more, when the two sequences are aligned. Preferably, the reference sequence is selected from SEQ ID No. 3, SEQ ID No. 6, SEQ ID No. 7, SEQ ID No. 9, SEQ ID No. 10, SEQ ID No. 12, SEQ ID No. 13, SEQ ID No. 15, SEQ ID No. 16, SEQ ID No. 18, SEQ ID No. 19, and SEQ ID No. 22.

As used herein, a “fragment” refers to a sequence containing less amino acids or nucleotides in length than the respective polypeptide sequence or nucleic acid sequence. Preferably, this sequence or fragment contains less than 90%, preferably less than 60%, in particular less than 30% amino acids or nucleotides in length than the respective polypeptide sequence or nucleic acid sequence, e.g. SEQ ID No. 3, SEQ ID No. 6, SEQ ID No. 7, SEQ ID No. 9, SEQ ID No. 10, SEQ ID No. 12, SEQ ID No. 13, SEQ ID No. 15, SEQ ID No. 16, SEQ ID No. 18, SEQ ID No. 19, and SEQ ID No. 22.

Further contemplated is a plasmid or a vector comprising a nucleic acid sequence of the invention.

For cloning of polynucleotides of the invention, the vector may be introduced into a host cell (autologous, allogeneic or heterologous) to allow replication of the vector itself and thereby amplify the copies of the polynucleotide contained therein. The cloning vectors of the invention may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

The term “autologous” refers to any material derived from the same individual to which it is later to be re-introduced.

The term “allogeneic” refers to any material derived from one individual which is then introduced to another individual of the same species, e.g., allogeneic T cell transplantation.

In certain aspects, the present disclosure provides isolated host cells, or population of cells, containing the vector or plasmid provided herein. The host cells, or population of cells, containing the vector or plasmid may be useful in expression or cloning of the polynucleotide contained in the vector. Suitable host cells can include, without limitation, oncolytic virus, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The vector or plasmid can be introduced to the host cell, or population of cells, using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector or plasmid of interest are well known in the art.

The present invention also contemplates compositions as well as pharmaceutical compositions.

In an aspect of the invention, the pharmaceutical composition of the invention comprises a therapeutically effective amount of a fusion protein described herein, a pharmaceutically acceptable carrier, a diluent and/or an excipient.

In an aspect of the invention, the pharmaceutical composition of the invention comprises a therapeutically effective amount of a plasmid or a vector described herein, a pharmaceutically acceptable carrier, a diluent and/or an excipient.

In an aspect of the invention, the pharmaceutical composition of the invention comprises a therapeutically effective amount of an isolated host cell, or population of cells described herein, a pharmaceutically acceptable carrier, a diluent and/or an excipient.

The pharmaceutical composition described above can further comprise an anti-cancer immunotherapy as described herein, preferably a cytokine therapy, more preferably a cytokine therapy selected from the group comprising IL-15, a fusion protein comprising IL-15, IL-15Ra and an immunoglobulin IgG Fc domain, or a combination of one or more thereof.

The pharmaceutical composition can further comprise, in addition to the fusion protein and/or anti-cancer therapy described herein, an ACT therapy selected from the group comprising TCR-T, CAR-T, TILs and NK cell therapy, or a combination of one or more thereof.

Also contemplated are methods of treatment and/or prevention of a cancer in a subject in need thereof.

In an aspect of the invention, the method of treatment and/or prevention of a cancer in a subject in need thereof comprises administering the pharmaceutical composition of the invention, alone or in combination with an anti-cancer immunotherapy as described herein, preferably a cytokine therapy, more preferably a cytokine therapy selected from the group comprising IL-15, a fusion protein comprising IL-15, IL-15Ra and an immunoglobulin IgG Fc domain, or a combination of one or more thereof.

The pharmaceutical composition can further comprise, in addition to the fusion protein and/or anti-cancer therapy described herein, an ACT therapy selected from the group comprising TCR-T, CAR-T, TILs and NK cell therapy, or a combination of one or more thereof.

In this particular aspect, the method of treatment and/or prevention of a cancer in a patient or subject comprises (i) removing and isolating immune cells, preferably native T cells, from said patient or subject, (ii) genetically engineering said T cells to encode a chimeric antigen receptor (CAR), a T cell receptor (TCR) or any other synthetic tumor targeting motif or antigen, (iii) expanding ex vivo into a larger population of engineered T cells, and (iv) reintroducing said engineered T cells, into the patient or subject. After the engineered T cells are reintroduced into the patient or subject, they mediate an immune response against cells expressing the tumor targeting motif or antigen described herein.

In one aspect the method of treatment and/or prevention of a cancer comprises (i) removing and isolating immune cells, preferably native T cells, from a patient or subject, or providing immune cells, preferably native T cells, (ii) genetically engineering said T cells with at least to encode a chimeric antigen receptor (CAR), a T cell receptor (TCR) or any other synthetic tumor targeting motif or antigen, (iii) expanding ex vivo into a larger population of engineered T cells, and (iv) reintroducing into the patient or subject.

Also contemplated is a method of enhancing ACT antitumor activity in a subject comprising administering the pharmaceutical composition of the invention.

Also contemplated is a kit for performing one or more methods according to the invention.

In one aspect, the kit comprises a composition or a pharmaceutical composition of the invention in one or more containers. Compositions can be in liquid form or can be frozen. Suitable containers for the compositions include, for example, bottles, vials, syringes, and test tubes. Containers can be formed from a variety of materials, including glass or plastic. The kit may further contain instructions that may include information or directions, drug quantity, composition, and so forth for the prescription.

The invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein. Various references are cited throughout this Specification, each of which is incorporated herein by reference in its entirety. The foregoing description will be more fully understood with reference to the following Examples.

### EXAMPLES

### IL-21 Enhances NK Cell Effector Function

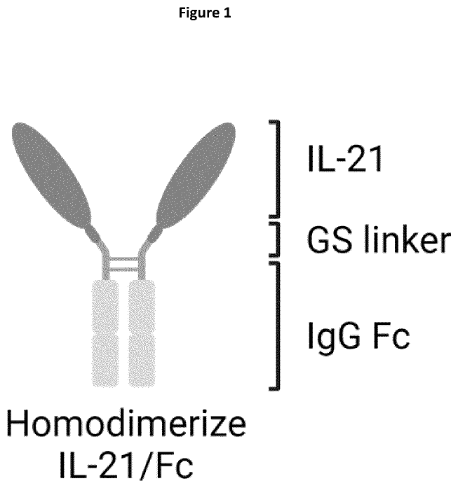

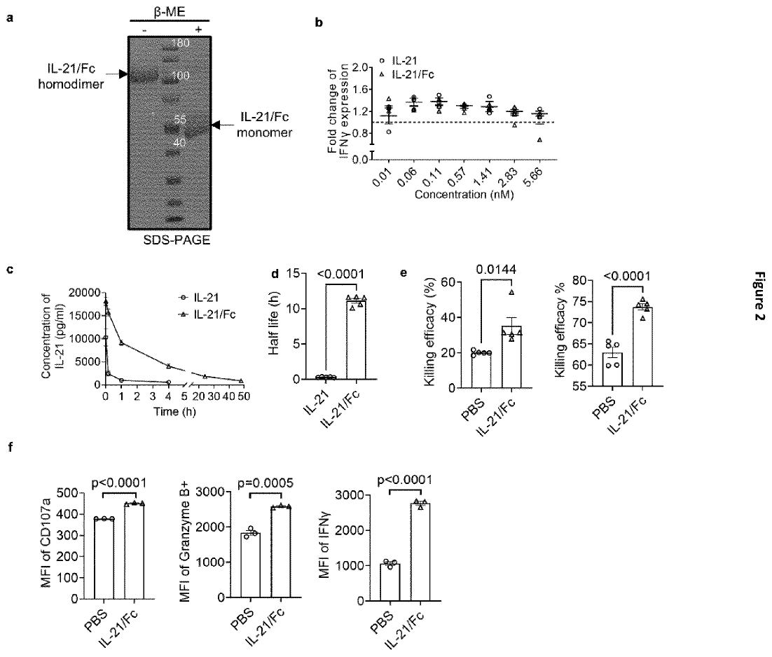

We firstly sought for investigating whether IL-21 could enhance NK cell effector function. We designed and produced a recombinant mouse IL-21 and mutant IgG1 Fc fusion protein (IL-21/Fc) (FIG. 1), which had longer half-life time compared to native IL-21 and retained comparable bioactivity (FIGS. 2a and 2b). The fused IL-21/Fc presented prolonged circulation by measurement of the IL-21 concentration in peripheral blood. Comparing to native mouse IL-21 at a small molecular weight and size, the half-life of IL-21/Fc was about 11.1 hours, which corresponds to an increase of 37-fold compared to native mouse IL-21 at 0.3 hours (FIGS. 2c and 2d). NK cells that isolated from mouse spleen were cultured in presence of IL-2 to activate NK cells in vitro and maintain their survival. After 6 days activation and proliferation, the activated NK cells were co-cultured with B16F10_β₂m cells (β₂ microglobulin knockout B16F10 cell line) and CT26_β₂m cells (β₂ microglobulin knockout CT26 cell line as described in Nicolai Christopher, J. et al. 2020) respectively at various E:T ratios to 0.5:1 in the presence of IL-21/Fc at 100 ng/ml. β₂ microglobulin is a component of MHC I molecules. These β₂ microglobulin knockout cell lines all have dysfunctional MHC I molecules and therefore cannot be recognized by T cells but are sensitive to NK cell mediated killing. The killing efficacy was determined by LDH assay after 5 hours incubation. It was clearly shown that IL-21/Fc is able to enhance the NK cells killing efficacy towards target cells significantly (FIG. 2e). More important, we noticed IL-21/Fc exerted NK cell killing function rapidly at a relatively lower E:T ratio of less than 1, which was similar to the real situation in the tumor microenvironment where less immune cells infiltrate. In addition, cytokines and degranulation capacity were measured through the CD107a expression and IFN-γ, granzyme B secretion. IL-21/Fc supported the NK cells antitumor activity through the promotion of degranulation capability and cytokines secretion (FIG. 2f).

### In Vivo Antitumor Effects of IL-21/Fc

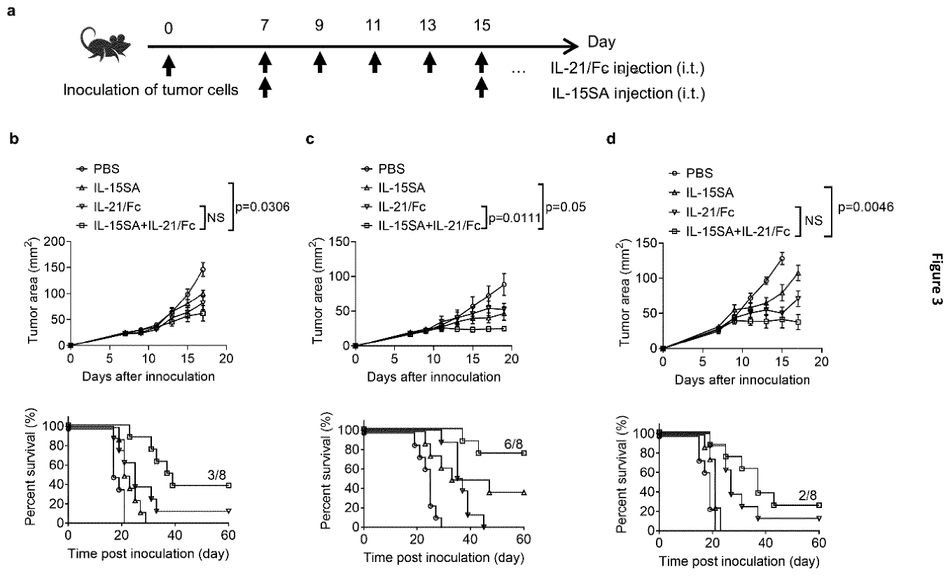

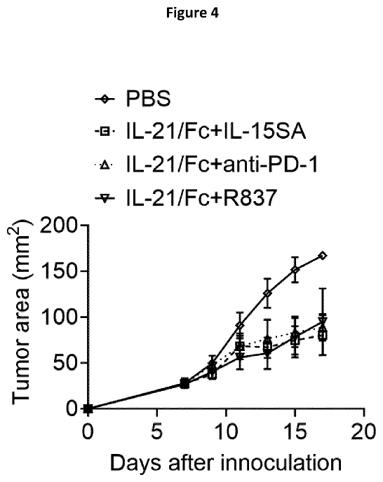

Next, we tried to investigate the in vivo antitumor effects of IL-21/Fc to validate whether it enhances NK cell function in cancer immunotherapy (FIG. 3a). We established the MHC I deficient tumor models to identify the NK cell antitumor activity since these tumors that lack of MHC I molecules are commonly escaped from immunosurveillance and lead to limited killing as poorly recognized by CD8+ T cells. We firstly evaluated the therapeutic efficacy in a highly aggressive and poorly immunogenic B16F10_β₂m knockout mouse melanoma model. Treatment with IL-21/Fc significantly contribute to control the tumor growth in early stage comparing to the PBS group, but the mice eventually succumbed with increased tumor burden. Considering the “cold” tumor immune microenvironment and to achieve a better therapeutic efficacy, we combined IL-15SA (IL-15 superagonist) together to increase NK cell infiltration and promote the IL-21/Fc antitumor potentials. IL-15 is a promising cytokine in NK cell translative studies since it shows robust effects on stimulating NK cell proliferation. Strikingly, treatment of IL-21/Fc and IL-15SA combination induced significant tumor regression and durable cures in 75% of mice bearing B16F10_β₂m tumors comparing to IL-21/Fc treatment alone (FIG. 3b). Comparing to B16F10_β₂m tumor model, IL-21/Fc itself abled to eliminate CT26_β₂m and RMA-S tumors but the durable efficacies are still limited, IL-15SA did not further regressed tumor growth compared to IL-21/Fc at the early period, but they substantially controlled the tumors growth and eradicated them which eventually led to prolonged mice survival (FIGS. 3c and 3d). To broaden the IL-21/Fc based immunotherapy, multiple combination therapies have been explored and tested on B16 melanoma tumor model. Combining IL-21 to currently used therapies such as immune checkpoint blockade antibody anti-PD-1, innate immune stimulator R837, showed a great suppression of tumor growth that is comparable to combination with IL-15SA, in the MHC I competent model (FIG. 4). Taken together, IL-21/Fc enhanced NK cell antitumor activity that is capable of controlling tumor growth. In addition, combination of IL-15SA further enhanced NK cell therapeutic efficacy in pre-established solid tumors.

NK Cells are the Dominate Immune Cell Subset that Contributed to Control the Tumor Growth

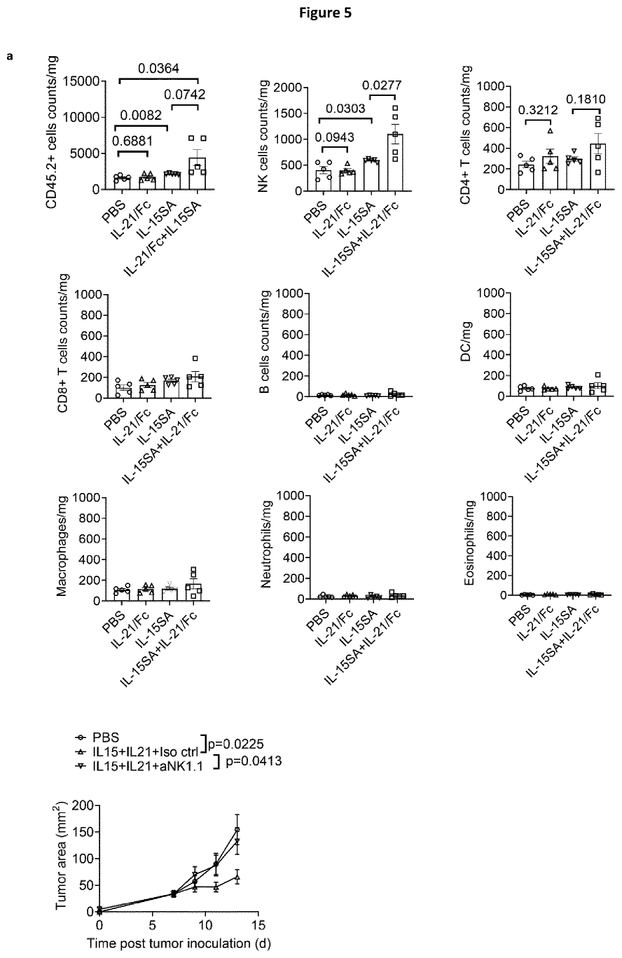

To validate whether NK cells were the dominate immune cell subset that contributed to control the tumor growth, we analyzed the tumor infiltrated CD45.2+ immune cells including NK cells, CD4+ T cells, CD8+ T cells, B cells, macrophages, dendritic cells, neutrophils and eosinophils from the CT26_β2m tumors. From the immune cell subsets changes with different treatment, IL-15 promoted the total CD45.2+ immune cells infiltration. In particular, NK cells account for the majority of infiltrated CD45.2+ immune cells in the tumor microenvironment in all groups, it was consistent to the typical feature of the tumor model that MHC I molecule deficient. As we expected, IL-15SA increased the NK cells infiltration by comparing to the PBS group, which probably due to the IL-15SA mediated stimulation and NK cells expansion. Treatment of IL-21/Fc in addition to IL-15SA, NK cells were further increased significantly. Although CD4+ T cells and CD8+ T cells were slightly increased under IL-15SA treatment, their absolute cell numbers were less than NK cells. Moreover, no matter what kind of treatment we gave, there was no significant difference of T cells among the four groups (FIG. 5a).

On the basis of the NK cells as the major immune cell subset in tumor microenvironment, we attempted to further confirm that NK played the dominant role in eradicating tumors. We kept the same schedule of IL-21/Fc and IL-15SA injection as we did in efficacy study but depleted the NK cells of the tumor bearing mice. The antitumor effects of IL-21/Fc and IL-15SA were completely abrogated upon we depleted the NK cells. The tumor growths of these mice were similar to the PBS group. In contrast, IL-21/Fc and IL-15SA shown greater antitumor efficacy and average survival to the mice additionally received isotype control antibody, which NK cells were remained (FIG. 5b). Tumor infiltrated immune cells analysis and efficacy study under specific immune cell depletion clearly revealed the dominant role of NK cells in the context of IL-21/Fc and IL-15SA combination therapy. Moreover, IL-21/Fc further favoured the increased number and enhanced cytolytic function of NK cells in the condition of enough NK cells infiltration that mediated by IL-15SA.

### Antitumor Activity of IL-21/Fc is Dependent on NK Cells in Tumor

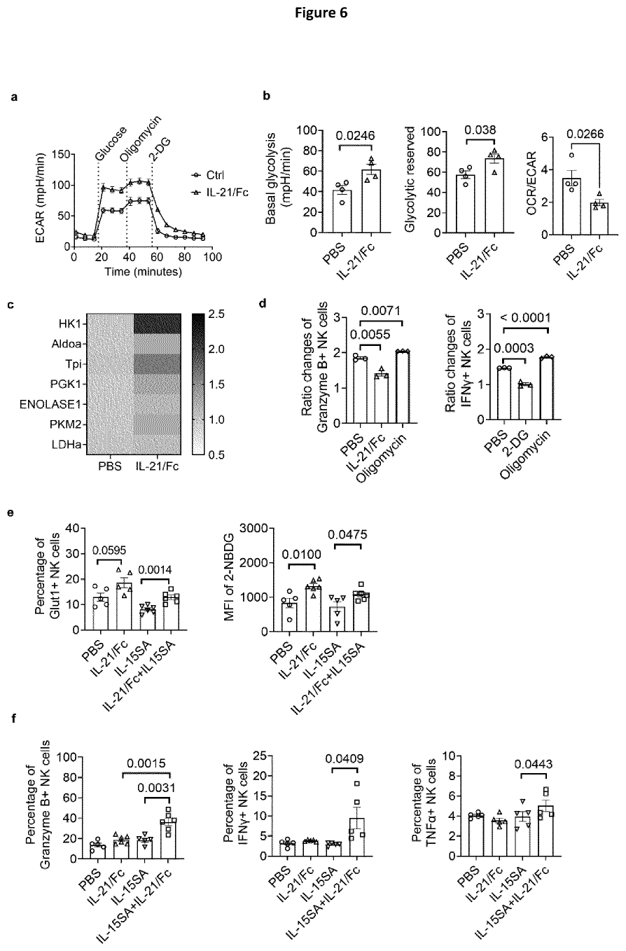

Considering that metabolism fulfils the biosynthetic and energy demands for cell fate and functions, we evaluated the metabolic changes of NK cells in presence of IL-21/Fc by seahorse assay. IL-21/Fc significantly enhanced the glycolysis of NK cells including the basal glycolysis level and reserved glycolytic capacity, but it did not highly alter the oxidative phosphorylation (OXPHOS) metabolism of NK cells, the metabolic profile of NK cells shifted to glycolysis in the presence of IL-21/Fc (FIGS. 6a and 6b). In addition, we identified the glycolysis associated gene and they were almost increased upon IL-21/Fc treatment (FIG. 6c). IL-21/Fc mediated NK cell functions were remained in presence of oligomycin, an OXPHOS metabolism inhibitor, but the function enhancement was abrogated while applying glycolysis inhibitor 2-Deoxy-D-glucose (2-DG) (FIG. 6d). These results suggested that IL-21/Fc worked on NK cell metabolism through enhancing glycolysis, and the metabolic activity changes was essential to NK cell function. To validate the IL-21/Fc mediated NK cell effector function enhancement attribute to the glycolytic metabolism, we measured the tumor infiltrated NK cell effector functions and glycolytic activity upon IL-21/Fc treated. The tumor infiltrated NK cells showed an increased expression of glucose transporter 1 (Glut1) and a higher uptake of 2-NBDG, a fluorescent glucose analog widely used to monitor glucose uptake in living cells (FIG. 6e). Correspondingly, tumor infiltrated NK cells that received IL-21/Fc showed enhanced effector function in the in the scenario of IL-15SA combination (FIG. 6f).

IL-21/Fc Enriches NK Cells with Promoted Stemness and Induce Durable Protection Again Tumor Rechallenge

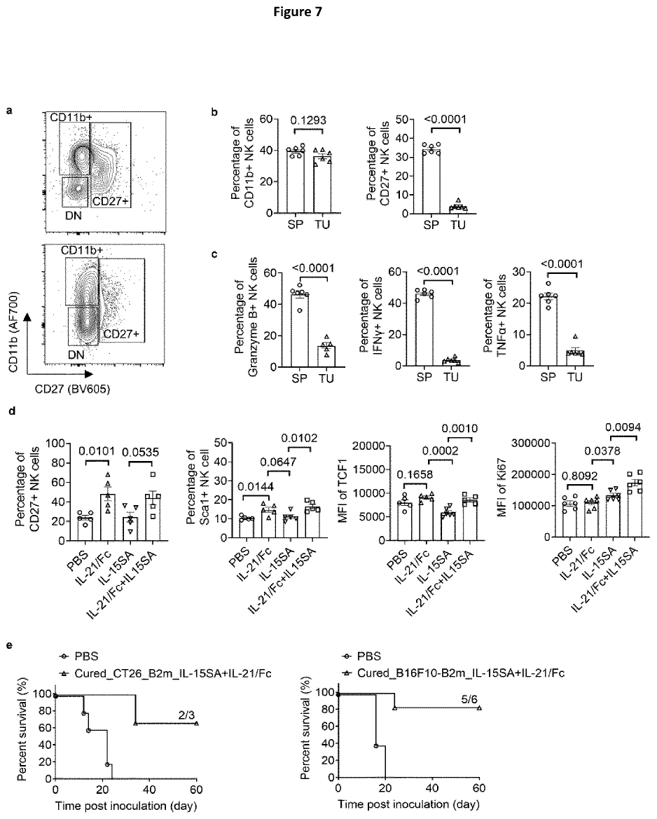

Except to the metabolic change, phenotypes changes were also investigated. By comparing the NK cells phenotype and functions differences between tumor and spleen, we observed a dramatic decrease of CD27+ NK population among tumor infiltrated NK cells, and the effector functions of tumor infiltrated NK cells were decreased as well. In contrast, the CD11b+ NK cell population remained almost unchanged (FIGS. 7a and 7b). CD27+ NK cells were reported with memory effects and stem-like features during infection (Kujur, W. et al. 2020), the decreased CD27+ NK in tumor microenvironment may explain the NK cells gradually lost functions in controlling tumor growth (FIG. 7c). Intriguingly, IL-21/Fc significantly increased CD27+ NK cell population in the TME. In addition, the expression of Sca1, TCF1 and Ki67 were increased in the NK cells receiving IL-21/Fc (FIG. 7d). Attributing to the increased CD27+ NK cells that have memory effects, the cured mice showed rejection to the secondary tumor implantation (FIG. 7e) (Venkatasubramanian, S. et al. 2017). Taken together, these results demonstrated that IL-21/Fc enhanced tumor infiltrated NK cell memory and stem-like features, which probably related to maintain the sustainable NK cell functions and tumor rechallenge.

### Human IL-21/Fc Promotes Human NK Cells Function and Glycolysis

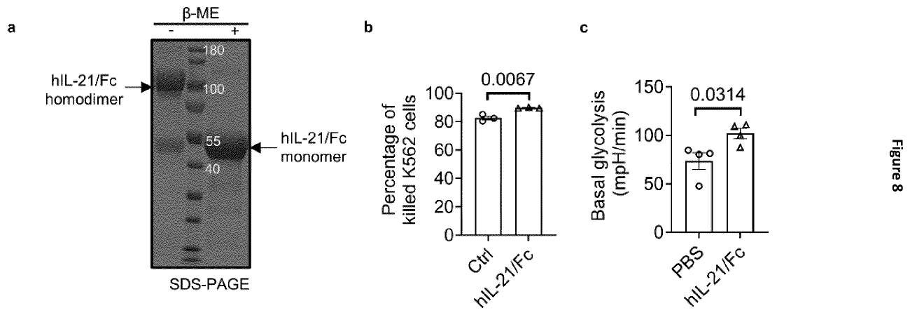

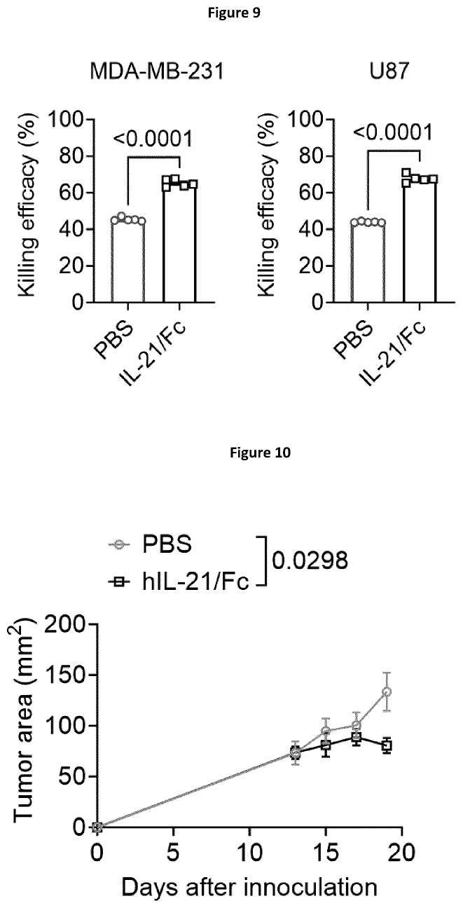

The effect of IL-21 on human NK cell were also evaluated. We produced fused human IL-21 (hIL-21/Fc) and tested its effects on NK92MI cells (FIG. 8a). hIL-21/Fc significantly enhanced the killing efficacy of NK92MI to K562 cells (FIG. 8b). Moreover, the NK92MI cells glycolysis metabolic activity was increased in presence of hIL-21/Fc (FIG. 8c). Except from validating the bioactivity of hIL-21/Fc on NK92MI cells, we also tested it on primary human NK cells that were isolated from human peripheral blood mononuclear cell (PBMC). PBMC derived NK (PBMC-NK) cells were previously activated by hIL-2 for 5 days. The activated PBMC-NK were cocultured with triple-negative breast cancer cell line MDA-MB-231 or glioblastoma cell line U87 at the E/T ratio to 0.5:1. The killing efficacy was determined by LDH assay after 5 hours incubation. Similar to its effects on NK92ML, hIL-21/Fc significantly increases the killing efficacy of PBMC-NK cells towards the two target cells (FIG. 9). To further explore hIL-21/Fc mediated antitumor activity in vivo, we established subcutaneous lymphoblast K562 tumor model on NSG mice. Mice received adoptive transfer of NK92MI cells as well as intratumoral injection of hIL-21/Fc. Additional injection of hIL-21/Fc significantly suppresses tumor growth indicating that IL-21/Fc enhances both NK cell antitumor effector functions and the antitumor efficacy considered to be dependent to NK cells (FIG. 10). The consistent results from human NK cells suggested the promising translative value of IL-21 in clinic.

In summary, we found IL-21/Fc induced substantial tumor regression and durable antitumor effects together with IL-15SA in multiple solid tumor models.

### REFERENCES

Wolf, N. K., Kissiov, D. U. & Raulet, D. H. Roles of natural killer cells in immunity to cancer, and applications to immunotherapy. Nat. Rev. Immunol. (2022).
Bald, T., Krummel, M. F., Smyth, M. J. & Barry, K. C. The NK cell-cancer cycle: advances and new challenges in NK cell-based immunotherapies. Nat. Immunol. 21, 835-847 (2020).
Li, Z.-Y. et al. The transcriptional repressor ID2 supports natural killer cell maturation by controlling TCF1 amplitude. J. Exp. Med. 218 (2021).
Bi, J. & Tian, Z. NK Cell Exhaustion. 8 (2017).
O'Brien, K. L. & Finlay, D. K. Immunometabolism and natural killer cell responses. Nat. Rev. Immunol. 19, 282-290 (2019).
Parrish-Novak, J. et al. Interleukin 21 and its receptor are involved in NK cell expansion and regulation of lymphocyte function. Nature 408, 57-63 (2000).
Nicolai Christopher, J. et al. NK cells mediate clearance of CD8+ T cell-resistant tumors in response to STING agonists. Science Immunology 5, eaaz2738 (2020).
Kujur, W. et al. Memory like NK cells display stem cell like properties after Zika virus infection. PLoS Pathog. 16, e1009132-e1009132 (2020).
Venkatasubramanian, S. et al. IL-21-dependent expansion of memory-like NK cells enhances protective immune responses against Mycobacterium tuberculosis. Mucosal Immunol. 10, 1031-1042 (2017).
Gorby C, Sotolongo Bellon J, Wilmes S, Warda W, Pohler E, Fyfe P K, Cozzani A, Ferrand C, Walter M R, Mitra S, Piehler J, Moraga I. Engineered IL-10 variants elicit potent immunomodulatory effects at low ligand doses. Sci Signal. 2020 Sep. 15
Saxton R A, Tsutsumi N, Su L L, Abhiraman G C, Mohan K, Henneberg L T, Aduri N G, Gati C, Garcia K C. Structure-based decoupling of the pro- and anti-inflammatory functions of interleukin-10. Science. 2021 Mar. 19
Armour K L, Clark M R, Hadley A G, Williamson L M. Recombinant human IgG molecules lacking Fcgamma receptor I binding and monocyte triggering activities. Eur J Immunol. 1999 August; 29(8):2613-24.
Zheng X X, Steele A W, Nickerson P W, Steurer W, Steiger J, Strom T B. Administration of noncytolytic IL-10/Fc in murine models of lipopolysaccharide-induced septic shock and allogeneic islet transplantation. J Immunol. 1995 May 15; 154(10):5590-600
Mumm J B, Emmerich J, Zhang X, Chan I, Wu L, Mauze S, Blaisdell S, Basham B, Dai J, Grein J, Sheppard C, Hong K, Cutler C, Turner S, LaFace D, Kleinschek M, Judo M, Ayanoglu G, Langowski J, Gu D, Paporello B, Murphy E, Sriram V, Naravula S, Desai B, Medicherla S, Seghezzi W, McClanahan T, Cannon-Carlson S, Beebe A M, Oft M. IL-10 elicits IFN7-dependent tumor immune surveillance. Cancer Cell. 2011 Dec. 13; 20(6):781-96
Karin M. Knudson, James W. Hodge, Jeffrey Schlom & Sofia R. Gameiro (2020) Rationale for IL-15 superagonists in cancer immunotherapy, Expert Opinion on Biological Therapy, 20:7, 705-709
Czajkowsky D M, Hu J, Shao Z, Pleass R J. Fc-fusion proteins: new developments and future perspectives. EMBO Mol Med. 2012 October; 4(10):1015-28.
Zalevsky J, Chamberlain A K, Horton H M, Karki S, Leung I W, Sproule T J, Lazar G A, Roopenian D C, Desjarlais J R. Enhanced antibody half-life improves in vivo activity. Nat Biotechnol. 2010 February; 28(2):157-9.
Vaccaro C, Zhou J, Ober R J, Ward E S. Engineering the Fc region of immunoglobulin G to modulate in vivo antibody levels. Nat Biotechnol. 2005 October; 23(10):1283-8.
Qiao J, Liu Z, Dong C, Luan Y, Zhang A, Moore C, Fu K, Peng J, Wang Y, Ren Z, Han C, Xu T, Fu Y X. Targeting Tumors with IL-10 Prevents Dendritic Cell-Mediated CD8+ T Cell Apoptosis. Cancer Cell. 2019 Jun. 10; 35(6):901-915.
Kontermann R E. Strategies for extended serum half-life of protein therapeutics. Curr Opin Biotechnol. 2011 December; 22(6):868-76.
Fan J, Shang D, Han B, Song J, Chen H, Yang J M. Adoptive Cell Transfer: Is it a Promising Immunotherapy for Colorectal Cancer? Theranostics. 2018 Nov. 10; 8(20):5784-5800.
Rosenberg S A, Restifo N P, Yang J C, Morgan R A, Dudley M E. Adoptive cell transfer: a clinical path to effective cancer immunotherapy. Nat Rev Cancer. 2008 April; 8(4):299-308.
Knudson K M, Hodge J W, Schlom J, Gameiro S R. Rationale for IL-15 superagonists in cancer immunotherapy. Expert Opin Biol Ther. 2020 July; 20(7):705-709.
Fujisaki H, Kakuda H, Shimasaki N, Imai C, Ma J, Lockey T, Eldridge P, Leung W H, Campana D. Expansion of highly cytotoxic human natural killer cells for cancer cell therapy. Cancer Res. 2009 May 1; 69(9):4010-7
Daher M, Melo Garcia L, Li Y, Rezvani K. CAR-NK cells: the next wave of cellular therapy for cancer. Clin Transl Immunology. 2021 Apr. 28; 10(4)

### SEQUENCE LISTING

human IL-21
SEQ ID NO: 1
QGQDRHMIRMRQLIDIVDQLKNYVNDLVPEFLPAPEDVETNCEWSAFSCFQKAQLKSANTGN
NERIINVSIKKLKRKPPSTNAGRRQKHRLTCPSCDSYEKKPPKEFLERFKSLLQKMIHQHLS
SRTHGSEDS
IgG1 Fc (corresponds to amino acids 99 to 330 of the hinge and Fc
domains of the full sequence of human IgG1)
SEQ ID NO: 2
EPKSADKTHTCPPCPAPEAAGGPSVFLFPPKPKDTLMISRTPEVTCVVVDVSHEDPEVKENW
YVDGVEVHNAKTKPREEQYNSTYRVVSVLTVLHQDWLNGKEYKCKVSNKALGAPIEKTISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
IL-21-Fc fusion protein 1
SEQ ID NO: 3
QGQDRHMIRMRQLIDIVDQLKNYVNDLVPEFLPAPEDVETNCEWSAFSCFQKAQLKSANTGN
NERIINVSIKKLKRKPPSTNAGRRQKHRLTCPSCDSYEKKPPKEFLERFKSLLQKMIHQHLS
SRTHGSEDSEPKSADKTHTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALGA
PIEKTISKAKGQPREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLSLSPGK
GGS linker
SEQ ID NO: 4
GGSGGSGGSGGSGGSGGSGGS
GGGGS linker
SEQ ID NO: 5
GGGGSGGGGSGGGGSGGGGS
Fc-IL-21 fusion protein 1
SEQ ID NO: 6
EPKSADKTHTCPPCPAPEAAGGPSVFLFPPKPKDTLMISRTPEVTCVVVDVSHEDPEVKENW
YVDGVEVHNAKTKPREEQYNSTYRVVSVLTVLHQDWLNGKEYKCKVSNKALGAPIEKTISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGKGGSGGSGGSGGSGGSG
GSGGSQGQDRHMIRMRQLIDIVDQLKNYVNDLVPEFLPAPEDVETNCEWSAFSCFQKAQLKS
ANTGNNERIINVSIKKLKRKPPSTNAGRRQKHRLTCPSCDSYEKKPPKEFLERFKSLLQKMI
HQHLSSRTHGSEDS
Fc-IL-21 fusion protein 1-2
SEQ ID NO: 7
EPKSADKTHTCPPCPAPEAAGGPSVFLFPPKPKDTLMISRTPEVTCVVVDVSHEDPEVKENW
YVDGVEVHNAKTKPREEQYNSTYRVVSVLTVLHQDWLNGKEYKCKVSNKALGAPIEKTISKA
KGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGKGGGGSGGGGSGGGGSG
GGGSQGQDRHMIRMRQLIDIVDQLKNYVNDLVPEFLPAPEDVETNCEWSAFSCFQKAQLKSA
NTGNNERIINVSIKKLKRKPPSTNAGRRQKHRLTCPSCDSYEKKPPKEFLERFKSLLQKMIH
QHLSSRTHGSEDS
IgG2 Fc (corresponds to amino acids 99 to 326 of the hinge and Fc
domains of the full sequence of human IgG2)
SEQ ID NO: 8
ERKCCVECPPCPAPPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPSSIEKTISKTKGQP
REPQVYTLPPSREEMTKNQVSLTCLVKGFYPSDIAVEWESNGQPENNYKTTPPMLDSDGSFF
LYSKLTVDKSRWQQGNVFSCSVMHEALHNHYTQKSLSLSPGK
IL-21-Fc fusion protein 2
SEQ ID NO: 9
QGQDRHMIRMRQLIDIVDQLKNYVNDLVPEFLPAPEDVETNCEWSAFSCFQKAQLKSANTGN
NERIINVSIKKLKRKPPSTNAGRRQKHRLTCPSCDSYEKKPPKEFLERFKSLLQKMIHQHLS
SRTHGSEDSERKCCVECPPCPAPPVAGPSVFLFPPKPKDTLMISRTPEVTCVVVDVSHEDPE
VQFNWYVDGVEVHNAKTKPREEQFNSTFRVVSVLTVVHQDWLNGKEYKCKVSNKGLPSSIEK
TISKTKGQPREPQVYTLPPSREEMTKNQVSLTCLVKGFYPSDIAVEWESNGQPENNYKTTPP
MLDSDGSFFLYSKLTVDKSRWQQGNVFSCSVMHEALHNHYTQKSLSLSPGK
Fc-IL-21 fusion protein 2
SEQ ID NO: 10
ERKCCVECPPCPAPPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPSSIEKTISKTKGQP
REPQVYTLPPSREEMTKNQVSLTCLVKGFYPSDIAVEWESNGQPENNYKTTPPMLDSDGSFF
LYSKLTVDKSRWQQGNVFSCSVMHEALHNHYTQKSLSLSPGKGGSGGSGGSGGSGGSGGSGG
SQGQDRHMIRMRQLIDIVDQLKNYVNDLVPEFLPAPEDVETNCEWSAFSCFQKAQLKSANTG
NNERIINVSIKKLKRKPPSTNAGRRQKHRLTCPSCDSYEKKPPKEFLERFKSLLQKMIHQHL
SSRTHGSEDS
IgG3 Fc
SEQ ID NO: 11
ELKTPLGDTTHTCPRCPEPKSCDTPPPCPRCPEPKSCDTPPPCPRCPEPKSCDTPPPCPRCP
APELLGGPSVFLFPPKPKDTLMISRTPEVTCVVVDVSHEDPEVQFKWYVDGVEVHNAKTKPR
EEQYNSTFRVVSVLTVLHQDWLNGKEYKCKVSNKALPAPIEKTISKTKGQPREPQVYTLPPS
REEMTKNQVSLTCLVKGFYPSDIAVEWESSGQPENNYNTTPPMLDSDGSFFLYSKLTVDKSR
WQQGNIFSCSVMHEALHNRFTQKSLSLSPGK
IL-21-Fc fusion protein 3
SEQ ID NO: 12
QGQDRHMIRMRQLIDIVDQLKNYVNDLVPEFLPAPEDVETNCEWSAFSCFQKAQLKSANTGN
NERIINVSIKKLKRKPPSTNAGRRQKHRLTCPSCDSYEKKPPKEFLERFKSLLQKMIHQHLS
SRTHGSEDSELKTPLGDTTHTCPRCPEPKSCDTPPPCPRCPEPKSCDTPPPCPRCPEPKSCD
TPPPCPRCPAPELLGGPSVFLFPPKPKDTLMISRTPEVTCVVVDVSHEDPEVQFKWYVDGVE
VHNAKTKPREEQYNSTFRVVSVLTVLHQDWLNGKEYKCKVSNKALPAPIEKTISKTKGQPRE
PQVYTLPPSREEMTKNQVSLTCLVKGFYPSDIAVEWESSGQPENNYNTTPPMLDSDGSFFLY
SKLTVDKSRWQQGNIFSCSVMHEALHNRFTQKSLSLSPGK
Fc-IL-21 fusion protein 3
SEQ ID NO: 13
ELKTPLGDTTHTCPRCPEPKSCDTPPPCPRCPEPKSCDTPPPCPRCPEPKSCDTPPPCPRCP
APELLGGPSVFLFPPKPKDTLMISRTPEVTCVVVDVSHEDPEVQFKWYVDGVEVHNAKTKPR
EEQYNSTFRVVSVLTVLHQDWLNGKEYKCKVSNKALPAPIEKTISKTKGQPREPQVYTLPPS
REEMTKNQVSLTCLVKGFYPSDIAVEWESSGQPENNYNTTPPMLDSDGSFFLYSKLTVDKSR
WQQGNIFSCSVMHEALHNRFTQKSLSLSPGKGGSGGSGGSGGSGGSGGSGGSQGQDRHMIRM
RQLIDIVDQLKNYVNDLVPEFLPAPEDVETNCEWSAFSCFQKAQLKSANTGNNERIINVSIK
KLKRKPPSTNAGRRQKHRLTCPSCDSYEKKPPKEFLERFKSLLQKMIHQHLSSRTHGSEDS
IgG4 Fc (corresponds to amino acids 99 to 327 of the
hinge and Fc domains of the full sequence of human IgG4)
SEQ ID NO: 14
ESKYGPPCPPCPAPEFEGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
IL-21-Fc fusion protein 4
SEQ ID NO: 15
QGQDRHMIRMRQLIDIVDQLKNYVNDLVPEFLPAPEDVETNCEWSAFSCFQKAQLKSANTGN
NERIINVSIKKLKRKPPSTNAGRRQKHRLTCPSCDSYEKKPPKEFLERFKSLLQKMIHQHLS
SRTHGSEDSESKYGPPCPPCPAPEFEGGPSVFLFPPKPKDTLMISRTPEVTCVVVDVSQEDP
EVQFNWYVDGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGK
Fc-IL-21 fusion protein 4
SEQ ID NO: 16
ESKYGPPCPPCPAPEFEGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYKTTPPVLDSDGSF
FLYSRLTVDKSRWQEGNVFSCSVMHEALHNHYTQKSLSLSLGKGGSGGSGGSGGSGGSGGSG
GSQGQDRHMIRMRQLIDIVDQLKNYVNDLVPEFLPAPEDVETNCEWSAFSCFQKAQLKSANT
GNNERIINVSIKKLKRKPPSTNAGRRQKHRLTCPSCDSYEKKPPKEFLERFKSLLQKMIHQH
LSSRTHGSEDS
Human serum albumin (HSA)
SEQ ID NO: 17
DAHKSEVAHRFKDLGEENFKALVLIAFAQYLQQCPFEDHVKLVNEVTEFAKTCVADESAENC
DKSLHTLFGDKLCTVATLRETYGEMADCCAKQEPERNECFLQHKDDNPNLPRLVRPEVDVMC
TAFHDNEETFLKKYLYEIARRHPYFYAPELLFFAKRYKAAFTECCQAADKAACLLPKLDELR
DEGKASSAKQRLKCASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGL
IL-21-HSA fusion protein
SEQ ID NO: 18
QGQDRHMIRMRQLIDIVDQLKNYVNDLVPEFLPAPEDVETNCEWSAFSCFQKAQLKSANTGN
NERIINVSIKKLKRKPPSTNAGRRQKHRLTCPSCDSYEKKPPKEFLERFKSLLQKMIHQHLS
SRTHGSEDSGGSGGSGGSGGSGGSGGSGGSDAHKSEVAHRFKDLGEENFKALVLIAFAQYLQ
QCPFEDHVKLVNEVTEFAKTCVADESAENCDKSLHTLFGDKLCTVATLRETYGEMADCCAKQ
EPERNECFLQHKDDNPNLPRLVRPEVDVMCTAFHDNEETFLKKYLYEIARRHPYFYAPELLF
FAKRYKAAFTECCQAADKAACLLPKLDELRDEGKASSAKQRLKCASLQKFGERAFKAWAVAR
LSQRFPKAEFAEVSKLVTDLTKVHTECCHGDLLECADDRADLAKYICENQDSISSKLKECCE
KPLLEKSHCIAEVENDEMPADLPSLAADFVESKDVCKNYAEAKDVFLGMFLYEYARRHPDYS
VVLLLRLAKTYETTLEKCCAAADPHECYAKVFDEFKPLVEEPQNLIKQNCELFEQLGEYKFQ
NALLVRYTKKVPQVSTPTLVEVSRNLGKVGSKCCKHPEAKRMPCAEDYLSVVLNQLCVLHEK
TPVSDRVTKCCTESLVNRRPCFSALEVDETYVPKEFNAETFTFHADICTLSEKERQIKKQTA
LVELVKHKPKATKEQLKAVMDDFAAFVEKCCKADDKETCFAEEGKKLVAASQAALGL
HSA-IL-21 fusion protein
SEQ ID NO: 19
DAHKSEVAHRFKDLGEENFKALVLIAFAQYLQQCPFEDHVKLVNEVTEFAKTCVADESAENC
DKSLHTLFGDKLCTVATLRETYGEMADCCAKQEPERNECFLQHKDDNPNLPRLVRPEVDVMC
TAFHDNEETFLKKYLYEIARRHPYFYAPELLFFAKRYKAAFTECCQAADKAACLLPKLDELR
DEGKASSAKQRLKCASLQKFGERAFKAWAVARLSQRFPKAEFAEVSKLVTDLTKVHTECCHG
DLLECADDRADLAKYICENQDSISSKLKECCEKPLLEKSHCIAEVENDEMPADLPSLAADFV
ESKDVCKNYAEAKDVFLGMFLYEYARRHPDYSVVLLLRLAKTYETTLEKCCAAADPHECYAK
VFDEFKPLVEEPQNLIKQNCELFEQLGEYKFQNALLVRYTKKVPQVSTPTLVEVSRNLGKVG
SKCCKHPEAKRMPCAEDYLSVVLNQLCVLHEKTPVSDRVTKCCTESLVNRRPCFSALEVDET
YVPKEFNAETFTFHADICTLSEKERQIKKQTALVELVKHKPKATKEQLKAVMDDFAAFVEKC
CKADDKETCFAEEGKKLVAASQAALGLGGSGGSGGSGGSGGSGGSGGSQGQDRHMIRMRQLI
DIVDQLKNYVNDLVPEFLPAPEDVETNCEWSAFSCFQKAQLKSANTGNNERIINVSIKKLKR
KPPSTNAGRRQKHRLTCPSCDSYEKKPPKEFLERFKSLLQKMIHQHLSSRTHGSEDS
mouse IL-21
SEQ ID NO: 20
HKSSPQGPDRLLIRLRHLIDIVEQLKIYENDLDPELLSAPQDVKGHCEHAAFACFQKAKLKP
SNPGNNKTFIIDLVAQLRRRLPARRGGKKQKHIAKCPSCDSYEKRTPKEFLERLKWLLQKMI
HQHLS
mouse Fc
SEQ ID NO: 21
EPRVPITQNPCPPLKECPPCAAPDLLGGPSVFIFPPKIKDVLMISLSPMVTCVVVAVSEDDP
DVQISWFVNNVEVHTAQTQTHREDYNSTLRVVSALPIQHQDWMSGKEFKCKVNNRALPSPIE
KTISKPRGPVRAPQVYVLPPPAEEMTKKEFSLTCMITGFLPAEIAVDWTSNGRTEQNYKNTA
TVLDSDGSYFMYSKLRVQKSTWERGSLFACSVVHEGLHNHLTTKTISRSLGK
mouse Fc-IL-21
SEQ ID NO: 22
EPRVPITQNPCPPLKECPPCAAPDLLGGPSVFIFPPKIKDVLMISLSPMVTCVVVAVSEDDP
DVQISWFVNNVEVHTAQTQTHREDYNSTLRVVSALPIQHQDWMSGKEFKCKVNNRALPSPIE
KTISKPRGPVRAPQVYVLPPPAEEMTKKEFSLTCMITGFLPAEIAVDWTSNGRTEQNYKNTA
TVLDSDGSYFMYSKLRVQKSTWERGSLFACSVVHEGLHNHLTTKTISRSLGKGGSGGSGGSG
GSGGSGGSGGSHKSSPQGPDRLLIRLRHLIDIVEQLKIYENDLDPELLSAPQDVKGHCEHAA
FACFQKAKLKPSNPGNNKTFIIDLVAQLRRRLPARRGGKKQKHIAKCPSCDSYEKRTPKEFL
ERLKWLLQKMIHQHLS

1. A method for treating cancer in a subject in need thereof, the method comprising administering to the subject a fusion protein or a pharmaceutical composition comprising the fusion protein, wherein said fusion protein comprises:
(i) an immunoglobulin IgG Fc domain or a human serum albumin (HSA) polypeptide, and
(ii) a polypeptide comprising a sequence of an Interleukin-21 (IL-21) polypeptide, or a fragment or a variant thereof,
wherein the IL-21 polypeptide, fragment, or variant thereof, is covalently fused to the N-terminus or the C-terminus of the IgG Fc domain or the HSA polypeptide via a linker.

2. The method according to claim 1, wherein the IgG Fc domain dimerizes with a second IgG Fc domain thereby forming a homodimer and wherein the second IgG Fc domain is covalently fused, via its N-terminus or C-terminus, to an IL-21 polypeptide, fragment, or variant thereof.

3. The method according to claim 1, wherein the IL-21 sequence comprises SEQ ID No. 1, a fragment, or a variant thereof.

4. The method according to claim 1, wherein the IL-21 variant is a biologically active derivative of an IL-21.

5. The method according to claim 1, further comprising administering, in addition to the fusion protein, an anti-cancer immunotherapy comprising ACT therapy, immune checkpoint blockade therapy, cytokine therapy, cancer vaccine therapy, bispecific antibody therapy, or a combination of one or more thereof.

6. The method according to claim 5, wherein ACT therapy comprises TCR-T, CAR-T, TILs, or NK cell therapy, or a combination of one or more thereof.

7. The method according to claim 5, wherein the cytokine therapy comprises GM-CSF, IFN gamma, IL-7, IL-10, IL-12, IL-15, a fusion protein thereof comprising (1) the cytokine and (2) an immunoglobulin IgG Fc domain or human serum albumin (HSA), or a combination of one or more thereof.

8. The method according to claim 5, wherein the cytokine therapy comprises IL-15, a fusion protein comprising IL-15, IL-15Rα and an immunoglobulin IgG Fc domain, or a combination of one or more thereof.

9. The method according to claim 8, wherein the fusion protein comprising IL-15, IL-15Rα and an immunoglobulin IgG Fc domain is IL-15 superagonist.

10. The method according to claim 6, wherein the NK cell therapy comprises induced or activated NK cells, iPSC-NK cells, CAR-NK cells, PBNK cells, or a combination of one or more thereof.

11. The method according to claim 10, wherein the NK cells are autologous or allogeneic NK cells.

12. The method according to claim 1, wherein the cancer is a solid cancer or a liquid cancer.

13. The method according to claim 12, wherein the solid cancer comprises lung cancer, breast cancer, ovarian cancer, cervical cancer, uterus cancer, head and neck cancer, glioblastoma, hepatocellular carcinoma, colon cancer, rectal cancer, colorectal carcinoma, kidney cancer, prostate cancer, gastric cancer, bronchus cancer, pancreatic cancer, urinary bladder cancer, hepatic cancer, brain cancer, skin cancer, or a combination of one or more thereof.

14. The method according to claim 1, wherein the cancer is an MHC-I expression altered cancer.

15. The method according to claim 5, wherein said immune checkpoint blockade therapy comprises an inhibitor comprising a CTLA-4 inhibitor, a TIM3 inhibitor, a PD-1 inhibitor, a LAG-3 inhibitor, a TIGIT inhibitor, a PD-L1 inhibitor, or a combination of one or more thereof.

16. The method according to claim 5, wherein the fusion protein increases the efficacy of the anti-cancer immunotherapy of an increase equal or superior to about 2% when compared to the efficacy of the anti-cancer therapy in the absence of the fusion protein.

17. The method according to claim 1, wherein the fusion protein increases the half-life and/or the number of NK cells of about 2% or more, when compared to the half-life and/or the number of NK cells in the absence of the fusion protein.

18. The method according to claim 1, wherein the Fc domain of an IgG is a silent Fc domain of an immunoglobulin (Ig) G.

19. The method according to claim 1, wherein the Fc domain of an IgG comprises a human IgG 1, a human IgG2, a human IgG3 and a human IgG4, or a fragment or a variant thereof.

20. The method according to claim 19, wherein the Fc domain of a human IgG comprises IgG1 Fc (SEQ ID No. 2), IgG2 Fc (SEQ ID No. 8), IgG3 Fc (SEQ ID No. 11), IgG4 Fc (SEQ ID No. 14), or a fragment, a variant, or a combination of one or more of these sequences.

21. The method according to claim 20, wherein the variant of SEQ ID No: 2 comprises at least one mutation selected from C220A, L234A, L235A and P329G.

22. The method according to claim 20, wherein the variant of SEQ ID No: 8 comprises at least one mutation selected from A330S and P331S.

23. The method according to claim 20, wherein the variant of SEQ ID No: 14 comprises at least one mutation selected from S228P and L235E.

24. The method according to claim 1, wherein the linker is a polypeptide linker.

25. The method according to claim 24, wherein the polypeptide linker consists of stretches of Gly and Ser residues (“GS” linker) or Gly-Gly and Ser residues (“GGS” linker) followed, or not, by one or more Arg residue (“R” residue”).

26. The method according to claim 1, wherein the fusion protein comprises SEQ ID No. 3, SEQ ID No. 6, SEQ ID No. 7, SEQ ID No. 9, SEQ ID No. 10, SEQ ID No. 12, SEQ ID No. 13, SEQ ID No. 15, SEQ ID No. 16, SEQ ID No. 18, SEQ ID No. 19, SEQ ID No. 22, or a fragment or a variant of any one of the sequences thereof.

27. A fusion protein comprising:
(i) an immunoglobulin IgG Fc domain or human serum albumin (HSA) polypeptide, and
(ii) a polypeptide comprising a sequence of an Interleukin-21 (IL-21) polypeptide, or a fragment or a variant thereof,
wherein the IL-21 polypeptide, fragment, or variant thereof, is covalently fused to the N-terminus or the C-terminus of the IgG Fc domain or the HAS polypeptide via a linker.

28. The fusion protein according to claim 27, wherein the IgG Fc domain dimerizes with a second IgG Fc domain thereby forming a homodimer and wherein the second IgG Fc domain is covalently fused, via its N-terminus or C-terminus, to an IL-21 polypeptide, fragment, or variant thereof.

29. The fusion protein according to claim 28, wherein the IL-21 sequence comprises SEQ ID No. 1, or a fragment or a variant thereof.

30. The fusion protein according to claim 27, wherein the IL-21 variant is a biologically active derivative of an IL-21.

31. The fusion protein according to claim 30, wherein the Fc domain of a human IgG comprises IgG1 Fc (SEQ ID No. 2), IgG2 Fc (SEQ ID No. 8), IgG3 Fc (SEQ ID No. 11), IgG4 Fc (SEQ ID No. 14), or a fragment, a variant, or a combination of one or more of these sequences.

32. The fusion protein according to claim 31, wherein the variant of SEQ ID No: 2 comprises at least one mutation selected from C220A, L234A, L235A and P329G.

33. The fusion protein according to claim 31, wherein the variant of SEQ ID No: 8 comprises at least one mutation selected from A330S and P331S.

34. The fusion protein according to claim 31, wherein the variant of SEQ ID No: 14 comprises at least one mutation selected from S228P and L235E.

35. The fusion protein according to claim 27, wherein the linker is a polypeptide linker.

36. The fusion protein according to claim 35, wherein the polypeptide linker consists of stretches of Gly and Ser residues (“GS” linker) or Gly-Gly and Ser residues (“GGS” linker) followed, or not, by one or more Arg residue (“R” residue”).

37. The fusion protein according to claim 27, wherein the fusion protein comprises SEQ ID No. 3, SEQ ID No. 6, SEQ ID No. 7, SEQ ID No. 9, SEQ ID No. 10, SEQ ID No. 12, SEQ ID No. 13, SEQ ID No. 15, SEQ ID No. 16, SEQ ID No. 18, SEQ ID No. 19, SEQ ID No. 22, or a fragment or a variant of any one of the sequences thereof.

38. A nucleic acid sequence encoding one or more fusion protein according to claim 27.

39. A plasmid or a vector comprising a nucleic acid sequence according to claim 38.

40. An isolated host cell, or population of cells, containing the vector or plasmid according to claim 39.

41. A pharmaceutical composition comprising i) a fusion protein according to claim 27, ii) a plasmid or a vector comprising the fusion protein or iii) an isolated host cell, or population of cells, comprising the plasmid or vector, and a pharmaceutically acceptable carrier, a diluent and/or an excipient.

42. The pharmaceutical composition according to claim 41, further comprising an anti-cancer immunotherapy.

43. The pharmaceutical composition according to claim 41, further comprising an ACT therapy comprising TCR-T, CAR-T, or TILs; or an NK cell therapy; or a combination of one or more thereof.

44. (canceled)

45. (canceled)

46. (canceled)

47. The pharmaceutical composition according to claim 41, wherein the host cell or population of cells are genetically engineered T cells encoding a chimeric antigen receptor (CAR), a T cell receptor (TCR), or any other synthetic tumor targeting motif or antigen.

48. (canceled)


SEQUENCE LISTING (22 sequences)

SEQ ID NO: 1 - protein, 133 aa, Homo sapiens [sequence omitted]

SEQ ID NO: 2 - protein, 232 aa, Homo sapiens
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 365 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSEPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 21 aa, synthetic construct
GGSGGSGGSGGSGGSGGSGGS

SEQ ID NO: 5 - protein, 20 aa, synthetic construct
GGGGSGGGGSGGGGSGGGGS

SEQ ID NO: 6 - protein, 386 aa, synthetic construct
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 7 - protein, 385 aa, synthetic construct
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 8 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - protein, 361 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 382 aa, synthetic construct
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGSGGSGGSGGSGGSGGSGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 11 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 12 - protein, 412 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 13 - protein, 433 aa, synthetic construct
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGGSGGSGGSGGSGGSGGSGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 14 - protein, 229 aa, Homo sapiens
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 362 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 383 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGSGGSGGSGGSGGSGGSGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 17 - protein, 585 aa, Homo sapiens [sequence omitted]

SEQ ID NO: 18 - protein, 739 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGSGGSGGSGGSGGSGGSGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 19 - protein, 739 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGGSGGSGGSGGSGGSGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 20 - protein, 129 aa, Mus musculus
HKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS

SEQ ID NO: 21 - protein, 238 aa, Mus musculus
EPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 22 - protein, 388 aa, synthetic construct
EPRVPITQNPCPPLKECPPCAAPDLLGGPSVFIFPPKIKDVLMISLSPMVTCVVVAVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGKGGSGGSGGSGGSGGSGGSGGSHKSSPQGPDRLLIRLRHLIDIVEQLKIYENDLDPELLSAPQDVKGHCEHAAFACFQKAKLKPSNPGNNKTFIIDLVAQLRRRLPARRGGKKQKHIAKCPSCDSYEKRTPKEFLERLKWLLQKMIHQHLS